(19) United States
(12) Patent Application Publication
(10) Pub. No.: US 2006/0246111 A1
(43) Pub. Date: Nov. 2, 2006

(54) Polysaccharides as ultrasound transmission media

(76) Inventor: Larry L. Smith, Lummi Island, WA (US)

Correspondence Address: ROBERT L. MCDOWELL, 1170 JACKSON HEIGHTS DR, WEBSTER, NY, 14580-9367, US

(21) Appl. No.: 11/406,874

(22) Filed: Apr. 19, 2006

Related U.S. Application Data
(60) Provisional application No. 60/672,809, filed on Apr. 19, 2005.

(51) Int. Cl. A61K 31/722; A61F 2/00

(52) U.S. Cl. 424/427; 514/54; 514/55

(57) ABSTRACT

Ultrasound couplants comprising polysaccharides, particularly sodium alginate and chitosan, and compatible substances for ultrasound imaging in applications that require either sterile or non-sterile forms of ultrasound transmission gels or liquids.

This application claims the benefit of U.S. Provisional Patent Application No. 60/672,809 filed Apr. 19, 2005, the entire disclosure of which is hereby incorporated by reference thereto.

### FIELD OF THE INVENTION

The present invention is directed toward ultrasound transmission and the medical use of acoustic coupling gels and fluids composed of polysaccharides which are used in ultrasound imaging, therapy and doppler based flow measurement.

### BACKGROUND OF THE INVENTION

Ultrasound, as used for medical applications, utilizes high frequencies, typically between 1 and 20 MHz for imaging and flow measurements, which are poorly transmitted by air and requires a coupling or conduction medium similar in acoustic properties to tissue, commonly a thick fluid or gel, to transfer the acoustic energy between the body and the electronics. The ultrasound coupling gel or fluid displaces air and fills contours between the piezoelectric transducer or “eye” of the instrument, which converts energy between electrical and acoustic, and the body into which the sound is being directed. This gel or fluid material, by nature of its physical and acoustic properties, serves as an ultrasound acoustic coupler between the body and the electronic transducer, thereby acoustically joining the two, so that the ultrasound based information developed, can freely pass back and forth between the body and the transducer. The gel or fluid material may also serve as a lubricant to aid in the introduction of a medical device used for imaging, such as endoscopes, into the body.

Because of the coupling effect, this media is commonly referred to as an ultrasound couplant, ultrasound gel, ultrasound transmission media or acoustic transmission media. Many fluids and water-based gels have been used as ultrasound couplants over the years. Early use of mineral oil was replaced by gels of water and acrylic based polymers such as CARBOPOL® (a registered trademark of BF Goodrich Specialty Chemicals) typical of those described in U.S. Pat. No. 4,002,221 to Buchalter, and also gels made from acrylic polymers and attached as coupling members to transducers such as are described in U.S. Pat. No. 4,459,854 to Richardson et al. as a method for improvement of perivascular blood flow measurement.

Polysaccharides, on the other hand are aqueous stabilizers and thickeners derived from natural sources such as marine plants, crustaceans, terrestrial plants and microbe fermentations that are commonly used in baked goods, beverages, dairy products, sauces and syrups, frozen foods and pharmaceutical applications.

Marine sources of linear polysaccharides are shells of crustaceans such as from the shells of crabs and shrimp, and various sea weeds. Algin (sodium alginate) is produced by alkali extraction of macrocystis pyrifera to form the sodium salt of a co-polymer of consisting of D-mannuronic acid and guluronic acid linked by a glycosidic linkage. The ratio of mannuronic/guluronic acid composition varies between species of kelp that in turn affects the molecular weight and the mechanical properties of the algin. Agar and another marine polysaccharide carrageenan, are derived from the red seaweed (Rhodophycae or Gracalaria), and generally consist of alternating segments of 3-linked-beta-D-galactpyranose and 4-inked-alpha-D-galactopyranose. The composition of agar differs slightly in that its structure contains sulfated galactose monomers. Agar is used as a gelling agent and as a stabilizer in pastry, confection and canned forms of meat products. The form and composition of the carrageenans vary with the species of seaweed and the extraction method, i.e. kappa carrageenan, is primarily derived from kappaphycus alvarezii, iota-carregeenan from eucheuma denticulatum and the lambda form from gigartina pistillata.

Of the seaweed based polysaccharides, algin and its derivatives have found numerous applications in food and pharmaceutical products. Commercial and pharmaceutical grades of alginate derivatives contain unacceptable levels of endotoxins that exclude their use in vivo due to their pyrogenic properties. The sodium alginate salt is now being ultra-purified by filtration processes and available from FMC Biopolymers, Novamatrix and others.

One particular marine substance, chitin, also known as acetylglucosamine, is second only to cellulose in abundance. The primary source is crab and shrimp shells that are treated with hydrochloric acid to remove impurities such as proteins, CaCO₃, lipids and pigments. By further processing chitin with sodium hydroxide, chitosan the deacetylated form of chitin is produced. Chitosan that has been sufficiently deacetylated, generally above 70%, is soluble in dilute acetic acid. Less pure forms of chitosan are acceptable as dietary supplements and water purification applications. However, for use in critical medical applications, such as gene therapy and implant, purification steps to remove endotoxins are required. Commercial and pharmaceutical grades of chitosan are further processed by micro and ultra-filtration to remove foreign debris and low molecular weight proteins that consist the endotoxins that cause pyrogenic reactions in humans.

U.S. Pat. No. 5,093,319 to Higham et al. teaches formation of cross-linked chitosan derivatives by treating films, produced by drying from aqueous solutions of chitosan compounds, with concentrated solutions of amino acids and borate, sulphate and phosphate ions. U.S. Pat. No. 5,420,197 to Lorenz et al. teaches production of gels consisting of open ring lactams, such as certain commercially available polyvinylpyrrolidones with chitosan to form films and membranes as dressings for applications related to burns, drug delivery and cosmetic face masks. Lorenz limits the scope of his teaching to such applications and does not teach use of such cross-linked hydrogels for medical ultrasound imaging applications.

A second source of polysaccharides is extracts and exudates of terrestrial plants and trees that are generally referred to as vegetable gums. These include substances such as guar, kayara, locust bean, cellulose, tragacanth, dextran, pectin and gum arabic, together with the lesser known gums, acacia, psyllium, gatti, pectin and konjak. These polysaccharide gums are generally composed of the monosaccharides D-galactose and D-mannose with less frequently occurring segments of xylose, glucose, pentose, hexuronic acid and L-rahmnose. With exception of grain based starches, agar, locust bean and guar gum constitute the greater portion of the market for gum based thickeners and stabilizers used in the food industry. Agar and guar gums are often used without blending; however, locust bean gum is often blended with xanthan and carrageenan to form more rigid gels. One or more of the linear polysaccharides are often combined to tailor the formulation to specific applications, i.e. blending of carrageenan and locust bean gum to add chewiness and slow down melting of frozen desserts and with guar gum to soften the texture produced by the action of carrageenan and locust bean gum.

A third class of linear polysaccharides is derived from microbial sources. Kelco Corporation commercialized xanthan gum produced by submerged aerobic fermentation technology using the bacterium Xanthomonas campestris in a nutrient medium. Xanthan gum is produced at the cell wall and is identical to that produced naturally by this bacterium on plants belonging to the cabbage family. In addition, Kelco has by similar technology, produced the bio-gums, gellan, rhamsan and welan. The pseudoplastic behavior and the relative insensitivity to temperature fluctuations on viscosity, tolerance to acids, alkali, salts and enzymes together with relatively high viscosity production at low concentrations, make xanthan a prime candidate for ultrasound scanning gels within the microbe produced polysaccharide class.

Polysaccharide cellulose derivatives represent a fourth class of polymers that efficiently thicken water and other solvents to form gels. These derivatives have applications as thickeners and suspending agents in foods and drug production, and various products including adhesives lubricants, cleaners, coatings and textiles. Representatives of this group include Carboxymethylcellulose (CMC), Methylhydroxypropylcellulose (MHPC), Hydroxyethylcellulose (HEC), modified starch, and propylene glycol alginate

By virtue of the chemical composition and molecular structure, most of the polysaccharide compounds produced from marine and terrestrial plants, and those of microbial origin are generally biocompatible and biodegradable in vivo. Polysaccharide derivatives, such as those produced by modification of cellulose may be biocompatible although in vivo biodegradability is questionable. By virtue of their origins, polysaccharides derived from natural sources contain high levels of endotoxins which may cause irritation or inflammation in vivo. Use of such polysaccharides in vivo requires purification to remove objectionable material such as proteins and remnants bacterial cell walls.

Current purification technologies such as ultra-filtration, electrophoresis and dialysis are used to remove protein and other impurities. Alginate purification processes such as those described by Clayton et al. in U.S. Pat. No. 5,529,913 and U.S. Pat. No. 6,372,244 to Antanavich et al. provide methods of sodium alginate purification such that proteins above 12,000 Daltons and other extrainous material is removed. U.S. Pat. No. 5,429,821 to Dorian et al. teach the use of ultra-purified sodium alginate that has been processed to produce high polymannurate gels that are non-toxic and non-fibrogenic when implanted in organs of the human body. Methods for alginate purification are complex and focused on objectives of producing materials that can be combined with cells such as for implantation of islet cells in the Islet of Langerhans for the treatment of diabetes and gene therapy without causing fibrotic or toxic reactions.

Gels of the most preferred embodiment of this inventive device use ultra-purified sodium alginate to produce gels that are in-vivo biocompatible and biodegradable for use in applications such as ultrasound imaging of organs and vessels during surgery, biopsy and procedures wherein patient safety is an issue. Such gels are useful in transceutanous ultrasound imaging for patients who are immuno-compromised.

### SUMMARY OF THE INVENTION

The invention is directed to ultrasound couplants comprising polysaccharides and compatible substances for diagnostic ultrasound imaging in applications that require sterile, in vivo biocompatible, biodegradable non-pyrogenic forms of ultrasound transmission gels for use during surgery and applications in vivo, and liquids and gels containing polysaccharides for transceutaneous imaging.

### DETAILED DESCRIPTION OF THE PREFERRED EMBODIMENTS

The present invention will now be discussed with reference to preferred embodiments which represent the invention by way of example only. Unless noted otherwise, all indicated percentages are weight percent.

The present invention contemplates the formulation and use of complex polysaccharides, in the form of gels and thickened liquids, as ultrasound transmission media and lubricants. Such complex polysaccharides include gums, starches, alginates and extracts and derivatives of marine plants, cellulose and chitin and derivatives, thereof. Formulations consist of these compounds used individually or in combination with other polysaccharides and compatible compounds to form gels or thickened liquids that provide acceptable low levels of artifact, distortion and attenuation of ultrasound energy. Such gels and thickened liquids can be formulated with various humectants, synthetic polymers, preservatives, antibiotics and curative agents, and in either sterile or non-sterile form.

Certain of the polysaccharides, for example, ultra-purified forms of chitosan and alginate are commercially available and useful for in-vivo applications. Sodium alginate and chitosan derivatives are commercially available from FMC Biopolymers, Philadelphia, Pa., and NovaMatrix. Whereas, complex polysaccharides such as guar, carragenen, xanthan, gellan, kayara, locust bean, cellulose, starch, tragacanth, dextrin, pectin, gum arabic, acacia, psyllium, gatti, pectin, konjak and lesser known vegetable gums and extracts are commercially available only in unpurified forms. Such polysaccharides currently appear to be of lesser interest for medical applications and have not received the benefits of intense purification and medical applications research as has sodium alginate and chitosan. As such, there is no commercial availability of these polysaccharides in ultra purified forms. However, should these materials be produced in the ultra-purified state, gels and thickened liquids produced from these materials using similar formulations to those of the preferred embodiments of the inventive device, could also function as ultrasound imaging couplants for in vivo applications.

Gels that are non-biocompatible can cause inflammation when in contact with organs, tissues and body fluids. Such contact can occur due to accidental gel spillage during ultrasound imaging assisted surgery and when performing ultrasound imaging, and ultrasound guided puncture procedures during which such gels can be carried into tissues from the puncture site.

Polysaccharides of terrestrial and marine origin were obtained from commercial sources and evaluated for use as ultrasound couplants by preparation of gels from the compounds solely and in combination with other polysaccharide compounds. Compounds tested included algin and chitosan derivatives, gums, and derivatives from bacterial fermentations such as Xanthan and Gellan to establish that many of the polysaccharides, although unpurified with respect to pyrogens can be produced and function as ultrasound couplants.

Common alginate derivatives include sodium, potassium and ammonium salts and the organic derivative propylene glycol alginate. Alginates consist of three polymer segments which vary in proportion according to species and location, being namely, D-manuronic, L-guluronic and a segment of alternating D-manuronic and L-guluronic acids. The proportion of these segments to each other determine the properties and functionality of the alginates, providing opportunities to manipulate M/G ratios which can influence fibrogenicity when implanted in the human body and to blend such alginates to provide various viscosity building potentials. To demonstrate the thickening and gel formation characteristics of alginates, sodium alginate salt from Sigma Aldrich, St Louis, Mo., was selected for formulation purposes as representative of both commercial grade and highly purified-forms of the salt.

A 2.5% solution of sodium alginate (Aldrich Lot # 09703 KU) was prepared in de-ionized (DI) water and the solution viscosity (centipoise) measured on a Brookfield LV Viscometer at 6 rpm with a #2 LVT spindle resulting in a viscosity of 720 cps. Similar formulations were prepared using 5 and 10% concentrations of Na Alginate. Viscosity results are as follows:

| % Na Alginate | Spindle & RPM | Viscosity (cps) |
|---|---|---|
| 2.5 | #2 LVT @ 6 RPM | 720 |
| 5.0 | #3 LVT @ 1.5 RPM | 19,200 |
| 10.0 | #4 LVT @ 0.3 RPM | 1,740,000 |

A 10% concentration of Na Alginate in water is similar to a spreadible paste. Whereas, 2.5 and 5% solutions are significantly lower in viscosity and tend to flow easily. Such gels have mechanical properties sufficient for use in medical ultrasound applications. However, the amount of polymer required to achieve ideal viscosity begins to negatively affect the rheology and flow characteristics. Greater thickening efficiency and gel tactile consistency can be achieved by cross-linking alginate salts with polyvalent cations such as zinc, aluminum, copper and calcium, which is most generally preferred. The reaction of calcium ions with alginate salt is generally represented as follows:
2NaAlg+Ca⁺⁺<>2CaAlg₂+2Na

This reaction which forms insoluble calcium alginate can also be applied to form gels with mechanical properties that are intermediate to fully cross-linked alginate salts by addition of calcium ions in quantities that are lower than the stoichiometric amount of 7.2% CaCl₂ based on the weight of sodium alginate. Cross-linking alginate salts creates gels of increased viscosity and gels that possess pseudoplastic behavior. Sodium alginate solutions that are fully cross-linked with polyvalent cations forms cohesive solids. Thick gels, suitable for use as ultrasound couplants can be produced by cross-linking sodium alginate with additions of calcium ions within the range of 15 to 30% of the stoichemetric amount. U.S. Pat. No. 6,309,380 to Larson et al. describes cross-linking of purified sodium alginate with Ca⁺⁺to produce an in vivo biocompatible film and vehicle for drug delivery in vivo, such as with the introduction of a stent into a blood vessel.

Cross-linking occurs rapidly with the addition cations such as Ca⁺⁺ which can result in clumping and non-homogenous alginate solutions. The rate at which cross-linking occurs can be controlled by use of sequestrants such as, di-calcium phosphate, tetrasodium pyrophosphate, sodium citrate and borates. These and other compounds sequester the calcium and subsequently slowly release it slowly, thus providing a measure of regulation to the rate at which cross-linking occurs. As the levels of sequestrants and calcium are varied, the resultant mechanical properties of cross-linked alginate gels can also vary from strong and rubbery to soft flowable gels such as could be used for medical ultrasound transmission couplants.

To demonstrate the production of cross-linked alginate gels suitable for use as ultrasound couplants, 200 gram samples of two formulations employing calcium concentrations of 15%, 30% and 45% of the stoichiometric amount were prepared.

### EXAMPLE 1

### 15% Stoichemetric Ca⁺⁺ Amount

| | | |
|---|---|---|
| Sodium Alginate (Aldrich #090703 KU) | 5.0 | grams |
| CaCl₂ (Spectrum #LI 0311) | 0.15 | gram |
| De-Ionized Water | 194.85 | grams |

### EXAMPLE 2

### 30% Stoichemetric Ca⁺⁺ Amount

| | | |
|---|---|---|
| Sodium Alginate (Aldrich #090703 KU) | 5.0 | grams |
| CaCl₂ (Spectrum #LI 0311) | 0.30 | gram |
| De-Ionized Water | 194.7 | grams |

### EXAMPLE 3

### 45% Stoichemetric Ca⁺⁺ Amount

| | | |
|---|---|---|
| Sodium Alginate (Aldrich #090703 KU) | 5.0 | grams |
| CaCl₂ (Spectrum #LI 0311) | 0.45 | gram |
| De-Ionized Water | 194.55 | grams |

As an alternative to the use of sequestrants and yet to avoid dumping of the alginate upon addition of calcium ions, in Examples 1, 2, 3, 50 grams of the DI water was reserved for solution and later addition to the alginate salt solution. Sodium alginate was first dissolved in approximately 150 grams of DI water with stirring until a smooth gel was formed, followed by addition of the CaCl₂ solution with continued stirring until an homogenous gel was produced. The samples were allowed to stand over-night followed by measurement of viscosity.

| Example # | % Na Alginate | % Ca Ion | % SA | Viscosity (cps)* |
|---|---|---|---|---|
| 1 | 2.5 | 0.075 | 15 | 56,200 |
| 2 | 2.5 | 0.15 | 30 | 90,500 |
| 3 | 2.5 | 0.225 | 45 | 172,000 |

*Brookfield LV Viscometer E Spindle @ 1.5 RPM Viscosity (cps)

As previously stated, the viscosity of an uncross-linked 2.5% sodium alginate solution was 720 cps which provides insufficient viscosity for efficient ultrasound imaging. Gels crosslinked at by addition of calcium ions at 15 and 30% of stoichemetric amounts were soft, smooth, pourable and produced. viscosities of 56,200 and 90,500 cps. Increase of the Ca⁺⁺ ion to the 45% level increased the viscosity in a linear fashion and created a stiffer thixotropic gel more rheologically suited for ultrasound coupling than Samples 1 & 2 above and aqueous solutions containing high concentrations of sodium alginate.

Alginates being of marine origin naturally contain elevated levels of bacterial remnants which when exposed to human tissue in vivo can cause inflammation and toxic reactions. These adverse reactions are the result of pyrogens, also known as endotoxins. Products made from algin derivatives are generally regarded as safe for oral ingestion and topical application, such as when used with transcutaneous ultrasound imaging. However, the risk associated with high endotoxin levels prevent or limit the use of alginates in vivo to ultra pure alginates. Such ultra pure (UP Alginate) is available from FMC Biopolymers and NovaMatrix companies as the sodium salt and finds use primarily for in vivo biocompatible implants and gene therapy. Use of alginates for ultrasound imaging in vivo also requires endotoxin levels below 0.5 endotoxin units (EU/ml) of the device. This is especially important should the ultrasound couplant be accidentally spilled and come into contact with tissues and organs.

Although cost is a commercial consideration that may affect marketability, such high purity alginate salts when prepared as solutions or as cross-linked gels, can perform as in vivo biocompatible, biodegradable ultrasound couplants.

Chitosan derived from chitin is also commercially available as various salts and derivatives that are used for various industrial applications such as water purification, dietary supplements, and low-endotoxin, ultra-purified forms for medical applications. High purity UP Chitosan for use as media for gene therapy and implant structures is available from FMC Biopolymer and NovaMatrix companies.

Samples of 90% deacetylated chitin, Lot # 9951 from Pangaea Sciences, Mississauga, ON, Canada, were used to evaluate gel formation and the potential use of chitosan as ultrasound coupling media for medical diagnostic imaging and therapy applications. To evaluate viscosity building and gel characteristics, solutions were prepared by dissolving 1, 2, and 3% quantities of chitosan in aqueous 2% acetic acid solutions. Resultant viscosities ranged from 10 cps to 1,100 cps,

A second approach to production of higher viscosity gels utilized the insolubility of chitosan in propylene glycol. The chitosan was first dissolved in 2% acetic acid, then added to propylene glycol and followed by viscosity determinations.

### EXAMPLE 4

| | | |
|---|---|---|
| Chitosan 90% DDA* | 2% | 2 grams |
| 2% Acetic Acid | 35% | 35 grams |
| Propylene Glycol | 63% | 63 grams |

Viscosity: Brookfield LV #2 LVT @ 3RPM - 1,900 cps

### EXAMPLE 5

| | | |
|---|---|---|
| Chitosan 90% DDA* | 3% | 3 grams |
| 2% Acetic Acid | 35% | 35 grams |
| Propylene Glycol | 62% | 62 grams |

Viscosity: Brookfield LV #2 LVT @ 3RPM - 5,860 cps
DDA*(Degree of Deacetylation)

When compared to water only based formulations, as previously described, the addition of propylene glycol to the formulation as a substitute for water provided enhanced viscosity, increased drying time and decreased tack while drying. While a thick gel is generally most preferred for ultrasound scanning procedures, the resultant viscosities of these samples provide sufficient thickness and resistance to flow to provide utility for use in applications where stiffer clean-breaking gels are not preferred.

The structure of chitosan is such that it can be ionic cross-linked to form gels with various mechanical properties that result as a function of the molecular weight of the chitosan, the structure of the cross-linker and degree of cross-linking. Chitosan and derivatives thereof, such as carboxymethyl (CM) chitosan can be cross linked with a broad range of compounds by direct addition to chitosan solutions in acidic environment, generally below pH 6. Examples of cross-linkers include amino acids, such as glutamic and aspartic, gluterahdehyde, acetic anhydride, acrylates and sulphate, borate and phosphate ions. In turn, Chitosan can be cross-linked with polyvinylpyrrlidone (PVP) to form gels.

Samples of Chitosan cross-linked with polyvinylpyrrilodone (PVP) were produced by first preparing a 3% solution of commercially available Chitosan (Spectrum) in an aqueous solution of 2% acetic acid followed by preparation of a 30% aqueous solution of ISP Povidone C-30 PVP. Three formulations were produced using 30% PVP/3% Chitosan in ratios of 4:1, 3:1 and 2:1. All formulations cross-linked to a degree that rendered a non-thixotropic rheology such that the gels exhibited cohesiveness thus limiting the ability to evenly spread the gels over a surface of the skin such as is preferred in medical ultrasound imaging applications. However, use of such chitosan cross-linked gels are useful for medical applications such as in the use of high intensity focused ultrasound (HIFU) for cautery and homeostasis.

Linear polysaccharides have also been derived from microbial sources. Kelco Corporation commercialized xanthan gum produced by aerobic submerged fermentation using the bacterium Xanthomonas campestris in a nutrient medium. Xanthan gum is produced at the bacteria cell wall and is identical to that produced naturally by this bacterium on plants belonging to the cabbage family. In addition, Kelco has by similar technology, produced the bio-gums, gellan, rhamsan and welan. The pseudoplastic behavior of these polysaccharides and the relative insensitivity to temperature fluctuations on viscosity, tolerance to acids, alkali, salts and enzymes together with relatively high viscosity production at low concentrations, are key factors to their use as medical ultrasound couplants.

Xanthan gum is commercially available in forms that aid rapid disbursement, slow and rapid hydration, brine tolerance and transparency. For purposes of evaluating the suitability of xanthan gums as gels for ultrasound scanning and coupling, Kelco's Keltrol T, an 80-mesh transparent powder was used. As a means of standardizing the formulations for comparison of gels formed by polysaccharides selected from each source group, an arbitrary polymer concentration of 2.5% for each of the gums and alginates was selected. Samples were prepared using de-ionized water as the solvent. Examples of polysaccharide formulations include xanthan gum, a blend of xanthan/locust bean gum, guar gum and gum tragacanth.

The Table below provides a summary of the viscosity produced by each of the example polysaccharide compounds.
Polysaccharide Gel Viscosity @ 2.5% Concentration in Water

Viscosity Brookfield LV

| Polymer Type | Spindle E @ 1.5 rpm |
|---|---|
| Xanthan-Keltrol T | 56,800 |
| Xanthan/Locust Bean Gum | 74,900 |
| Guar Gum | 113,600 |
| Gum Tragacanth | 50,400 |

The thickening capabilities of polysaccharide gums and their rheology provide utility for use as ultrasound transmission media. These four gums represent polysaccharides that are derivatives of terrestrial plants and trees, bacterial fermentation and synthetics produced by reaction with organic compounds. The variety of gels and thickened fluids of utility for use as ultrasound couplants, is not limited to simple one-component gels, but can consist of blends of one or several different polysaccharides to modify gel texture, strength, rheology and mechanical properties desired for the intended product or application. For example, combinations of xanthan with the galactomannans such as locust bean, guar and cassia gums to form high viscosity gels, and combinations of gellan and xanthan gums with carboxymethylcellulose are compounded for use in water control.

The polysaccharide starch, a major thickening agent widely used in industrial and food applications is combined with gellan gum and xanthan to form easily broken light textured gels for food applications. Polysaccharides can also be bended with other non-polysaccharide materials such as the protein thickener gelatin, humectants such as propylene glycol and glycerin, salts, organic solvents, surfactants and medications which speak to the broad range of applications and products provided by the versatility of these compounds.

The foregoing discloses not only the versatility of the linear polysaccharides for a wide range of applications in the food industry and pharmaceutical compounding but also their use as couplants for transceutanous medical ultrasound applications.

In particular, purified forms of alginate salts and chitosan, wherein low endotoxin levels have been produced, can be formulated as biocompatible and biodegradable gels for in vivo ultrasound applications, such as medical ultrasound imaging of tissues and vital organs during surgery.

While the invention has been described with reference to preferred embodiments it is to be understood that the invention is not limited to the particulars thereof. The present invention is intended to include modifications which would be apparent to those skilled in the art to which the subject matter pertains without deviating from the spirit and scope of the appended claims.

What is claimed is:

1. An ultrasound couplant in the form of a thickened liquid or gel, said ultrasound couplant comprising 1-10 wt. % of one or more polysaccharides and the balance water.

2. The ultrasound couplant of claim 1 wherein said one or more polysaccharides include endotoxins in an amount sufficiently small whereby said ultrasound couplant is in vivo biocompatible.

3. The ultrasound couplant of claim 1 wherein said one or more polysaccharides comprises sodium alginate.

4. The ultrasound couplant of claim 1 wherein said one or more polysaccharides comprises chitosan.
